(19) United States
(12) Patent Application Publication
(10) Pub. No.: US 2008/0217332 A1
(43) Pub. Date: Sep. 11, 2008

(54) APPARATUS AND METHOD FOR COLLECTION OF SHARP MEDICAL DISPOSABLES

(76) Inventors: Gennady Kleyman, Brooklyn, NY (US); Alexander Merson, Brooklyn, NY (US)

Correspondence Address: THE FARRELL LAW FIRM, P.C., 333 EARLE OVINGTON BOULEVARD, SUITE 701, UNIONDALE, NY, 11553, US

(21) Appl. No.: 12/041,700

(22) Filed: Mar. 4, 2008

Related U.S. Application Data
(60) Provisional application No. 60/905,016, filed on Mar. 5, 2007.

(51) Int. Cl. B65D 43/26; A61B 17/06; B65D 47/00; B65D 43/02; B65D 25/28

(52) U.S. Cl. 220/211; 206/366; 220/86.1; 220/260; 220/752

(57) ABSTRACT

Disclosed is a method to avoid and apparatus for avoiding sticking by a medical sharp following a medical procedure by creating an immediately available disposal point prior to the medical procedure by positioning a mouth of a sharps collection apparatus adjacent to a patient prior to the medical procedure to allow the caregiver to dispose of the medical sharp by pushing the sharp into the mouth of the apparatus, thereby eliminating excess motion by the caregiver during the medical procedure.

### PRIORITY

This application claims priority to U.S. Provisional Patent Application Ser. No. 60/905,016, filed with the U.S. Patent and Trademark Office Mar. 5, 2007, the contents of which are incorporated herein by reference.

### BACKGROUND

1. Field of the Invention

The present invention relates to an apparatus and method for safe disposal of used needle syringes, a/k/a medical sharps, and, in particular, to an apparatus and method to facilitate an expedited disposal of sharps utilized in hospital and medical facilities.

2. Description of the Related Art

Numerous conventional safety devices have been developed to prevent accidental needle-stick injuries to health care workers when handling used hypodermic needles during medical procedures. Certain conventional syringes retract the needle into a syringe barrel. Other conventional syringes enclose the needle within a cover. However, such conventional devices require manipulation of the syringe by the medical worker before the syringe can be placed into a medical sharp waste container. Such manipulation is time consuming and often overlooked or delayed during the time-intensive demands of medical treatment.

U.S. Pat. No. 7,250,038, which is incorporated by reference herein, to Simpson et al. discloses a fixed needle syringe with a syringe barrel having a modified distal portion that accepts a needle protective housing. In the device of Simpson et al., a predetermined torque must be to either a collar or housing, which is pivoted to cover the needle extending from the needle hub of the syringe. Similarly, U.S. Pat. No. 7,241,277, which is incorporated by reference herein, to Rimlinger et al., discloses a syringe safety device having a protective sheath in which the syringe body slides between injection and safe positions. U.S. Pat. No. 6,929,126, which is incorporated by reference herein, to Herbert discloses a syringe disposal device for encasing a single syringe.

However, these conventional safety devices require a degree of manipulation, often requiring forcing of a cover onto the needle by pushing with a finger or thumb, or pulling the plunger out to retract the needle into the syringe barrel. A caregiver is often unable to pause during a medical procedure to attend to such manipulation, and an act of forcing can result in an unintended needle-stick injury.

U.S. Pat. No. 5,303,822, which is incorporated by reference herein, to Wengyn et al. discloses a receptacle to support and retain a of used hypodermic needle and syringe combinations after use. Wengyn et al. discloses an outer receptacle support with a centrally disposed canister recess and a plurality of peripherally disposed substantially cylindrical liquid retaining tube recesses formed therein to selectively receive and support an inner disinfectant canister and a plurality of substantially cylindrical liquid retaining tubes respectively wherein the inner disinfectant retaining canister includes a substantially cylindrical tube to operatively house a disinfectant solution therein and a penetrable membrane to receive the used hypodermic needles there through to support and retain the used hypodermic needles in the disinfectant solution for subsequent disposal of the used hypodermic needle and syringe combinations.

Also see U.S. Pat. No. 4,035,911, which is incorporated by reference herein, to Nethercutt et al. proposes an alternative solution of providing a hand operated syringe destroyer for severing the needle at its hub.

U.S. Pat. No. 5,673,790, which is incorporated by reference herein, to Shramm discloses a disposal device for safe containment of contaminated medical sharps prior to permanent disposal. Shramm provides a hollow housing and sharps-anchoring material with a flange to stabilize the disposal device on a flat surface, to provide a gripping surface for one-handed insertion of a contaminated medical sharp.

Canister receptacles have also been proposed. See U.S. Pat. Nos. 4,890,733, 4,571,281, and 4,494,652, Pub. No. 2005/103662 A1, and PCT Pubs WO 91/10458 and WO 91/01920, the contents of each of which is incorporated by reference herein. However, such canister receptacles are cumbersome, inconvenient and not adaptable to the dynamics of the medical environment.

A paramount concern is to allow the caregiver to maintain the concentrated effort necessary to provide proper medical attention. However, conventional devices and methods distract the caregiver. In addition, use of certain conventional devices during a busy medical procedure can result in an obstructed view of the syringe or of the needle tip, which can result in an unintended needle stick. In addition, such conventional devices are typically significantly more expensive than traditional syringes.

A significant limitation to the usefulness of conventional devices is the need for a caregiver to divert his/her attention from the procedure in order to properly manipulate the conventional safety device. In medical procedures, a needle stick will often occur after removal of the medical sharp from the patient, due to a loss of concentration following the stress of properly attending to an insertion of the medical sharp into the patient.

The present invention overcomes the restrictions of conventional methods and devices to provide eliminate any chance of accidental needle-stick injures.

### SUMMARY OF THE INVENTION

An aspect of the present invention overcomes the shortcoming of conventional devices and provides the following advantages.

An aspect of the present invention provides a method to avoid sticking by a medical sharp by creating an immediately available disposal point prior to a medical procedure by positioning a mouth of a sharps collection apparatus adjacent to a patient prior to the medical procedure; wherein pushing the medical sharp into the mouth disposes of the medical sharp, to eliminate excess motion by the caregiver during the medical procedure.

A further aspect of the present invention allows the caregiver to operate a swing handle to rotate a mouth lid to allow the sharp to fall into a chute, wherein a carriage of the disposal apparatus transports the sharp.

Another aspect of the present invention provides an apparatus for quarantine of medical waste while allowing the mouth of the apparatus to be freely positioned regardless of whether a gravity assist is present to carry the sharp through the chute, the apparatus includes a mouth, a flexible chute, and a collection container, wherein the mouth has a opening relatively larger than the chute and the collection container is removable. In a preferred embodiment, a spring returns a lid of the mouth to a closed position and a handle operates the mouth lid to close the chute.

An aspect of the present invention provides a further advantage of a mechanical conveyance that includes a carriage for transport of a sharp, a carriage guide coupled to the chute to guide the carriage from the mouth to the container, as well as pulleys, a cable and a motor for moving the carriage along the carriage guide between receiving and discharge positions. In a preferred embodiment, cable holders are mounted on the carriage, carriage rollers are mounted on shafts within the carriage guide, a carriage lid rotatably attaches to the carriage via a carriage lid shaft and a torsion spring biases the carriage lid in a closed position. In a preferred embodiment, release of the handle returns mouth lid to a closed position and activates the motor to drives the cable to move the carriage to a discharge position. When the carriage lid reaching the cam, the carriage lid shaft rotates to open the lid to deposit the sharp in the collection container.

### BRIEF DESCRIPTION OF THE DRAWINGS

For a better understanding of the invention as well as other objects and further features thereof, reference is made to the following detailed description to be read in conjunction with the accompanying drawings, wherein:

FIG. 1 is a perspective view showing use by a medical caregiver of an embodiment of the present invention;

FIG. 2 is a perspective view of the apparatus of the present invention;

FIG. 3 is a front view of the apparatus of the present invention;

FIG. 4 is a side view of the apparatus of the present invention;

FIGS. 5 and 6 are views of area IV of the apparatus of FIG. 4;

FIG. 7 is an enlarged view of area VI of the apparatus of FIG. 4;

FIG. 8 is a perspective view of a second embodiment of the invented apparatus;

FIGS. 9 and 10 are cross-sectional views of the apparatus of FIG. 8 showing initial and final positions of a needle inserted therein;

FIGS. 11 and 12 show a detailed operation of Section VII-VII of FIG. 9;

FIG. 13 shows Section VIII-VIII of FIG. 10 with a mouth lid in an open position; and

FIG. 14 is a detailed view of the carriage and a motor of the apparatus of FIGS. 8-13.

### DETAILED DESCRIPTION OF THE PREFERRED EMBODIMENTS

The following detailed description of a preferred embodiment of the invention is provided in reference to the accompanying drawings. In describing the invention, explanation about related functions or constructions that are known in the art will be omitted for the sake of clearness in understanding the concept of the invention, as such would obscure the invention with unnecessary detail.

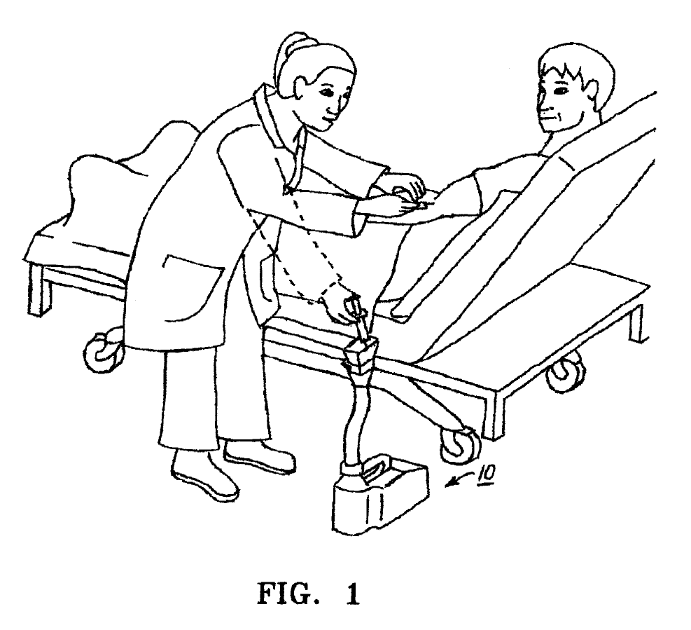

FIG. 1 shows the ease and convenience that is provided to the medical caregiver by the inventive apparatus 10. That is, use of apparatus 10 allows the caregiver to immediately dispose of sharp medical waste while remaining in close contact to the patient. In a preferred embodiment, the apparatus 10 can be attached to a patient's bed, thereby facilitating procedures that are carried out while a patient is bed ridden.

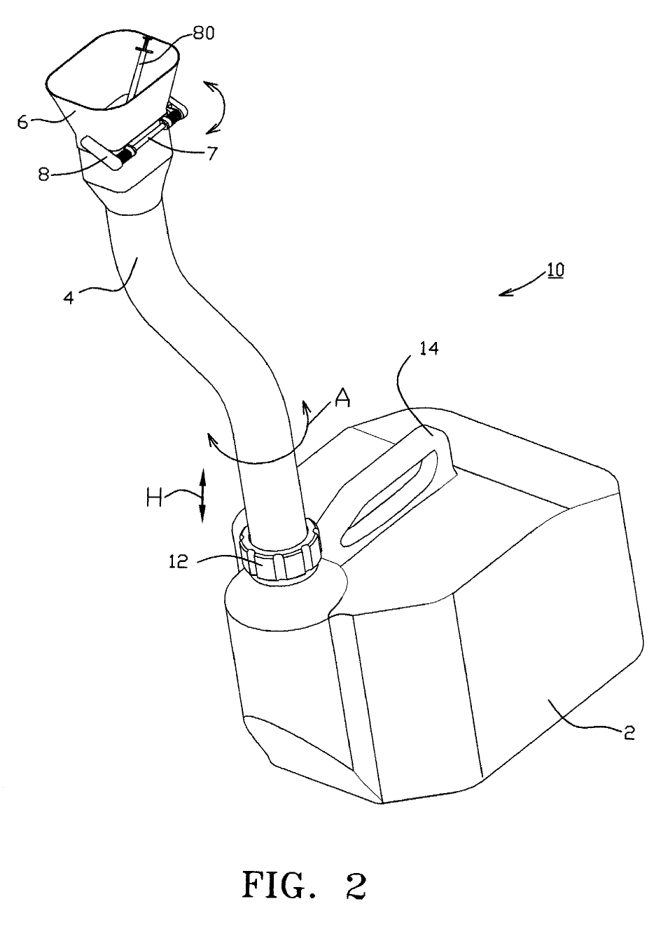

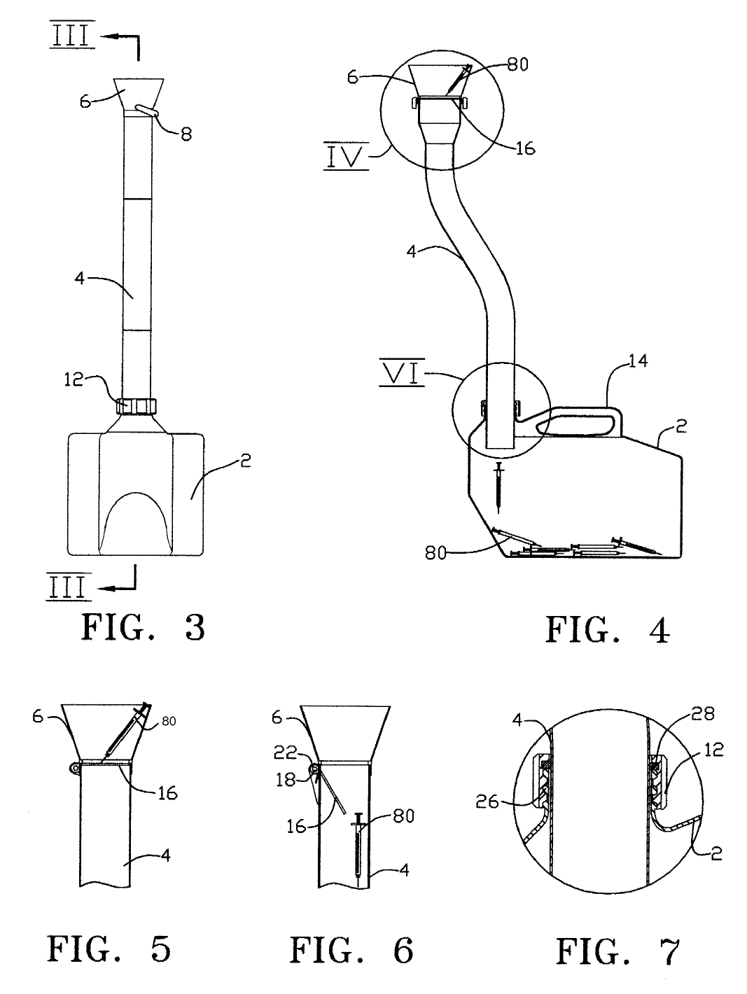

Flexible chute 4 (FIG. 2) is rotatable about axis A to allow the caregiver to precisely locate 6 to conveniently dispose of medical sharps, also referred to as sharp medical waste 80 herein, immediately after use thereof.

In a first preferred embodiment of that inventive apparatus a removable collection container 2 is provided that attaches via a joint 12, creating seal 28, to flexible chute 4, which is vertically adjustable by loosening and tightening of the joint 12 and is horizontally adjustable by rotation of the chute 4 around axis ‘A’, allowing for precise location of mouth 6, which is provided at a distal end of chute 4, close to a medical procedure.

As shown in FIG. 1, it is preferable to position mouth 6 close to a patient prior to the caregiver administering a shot or taking a blood sample. The flexibility of positioning allows the caregiver/practitioner to avoid an unintended sticking by a contaminated needle. That is, by creating an immediately disposal point prior to medical procedure, excess motion by the caregiver during medical procedure is eliminated. For example, use of conventional canister devices requires a caregiver to walk across a room with a used sharp, thereby diverting the attention of the caregiver from the patient. Also, when a caregiver administers a procedure from a seated position, the caregiver often remains in a seated position and rolls their chair a conventional canister disposal device. It will be appreciated that rolling across a room with a contaminated sharp can readily result in an unintended sticking.

The inventive apparatus preferably includes a mouth 6 having a opening relatively larger than chute 4, allowing the caregiver to easily and quickly dispose of the sharp medical waste by simply pushing a sharp medical waste 80 into the mouth 6, eliminating any accidental needle prick/sticking. A spring 18 is preferably provided to return a lid 16 to a closed position.

In a further embodiment, a handle 8 is provided to operate a mouth lid 16 (FIGS. 5-6) that controls what falls into chute 4. In this embodiment, when the sharp medical waste 80 is placed into mouth 6, handle 8 is operated and the sharp 80 falls through chute 4 into collection container 2. A spring 18 is also preferably provided so that release of handle 8 returns lid 16 to a closed position.

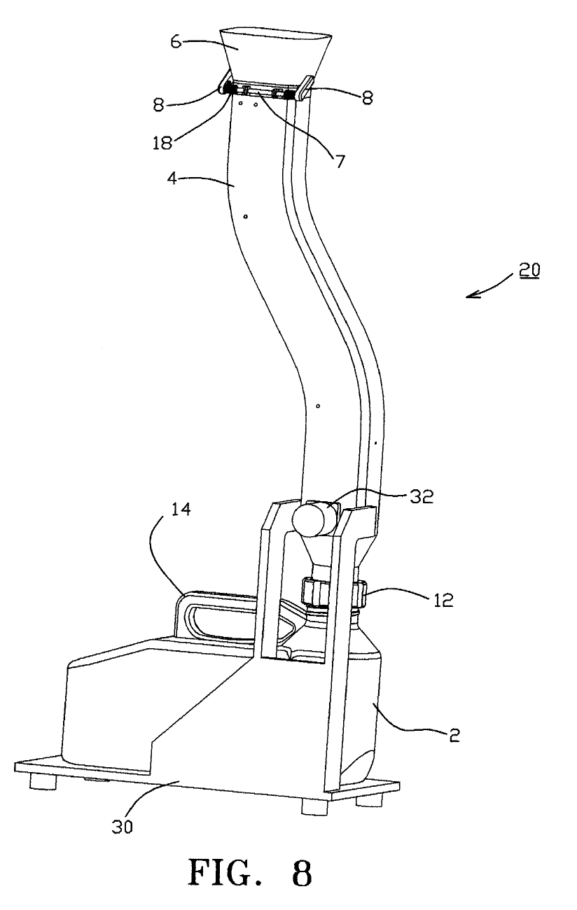

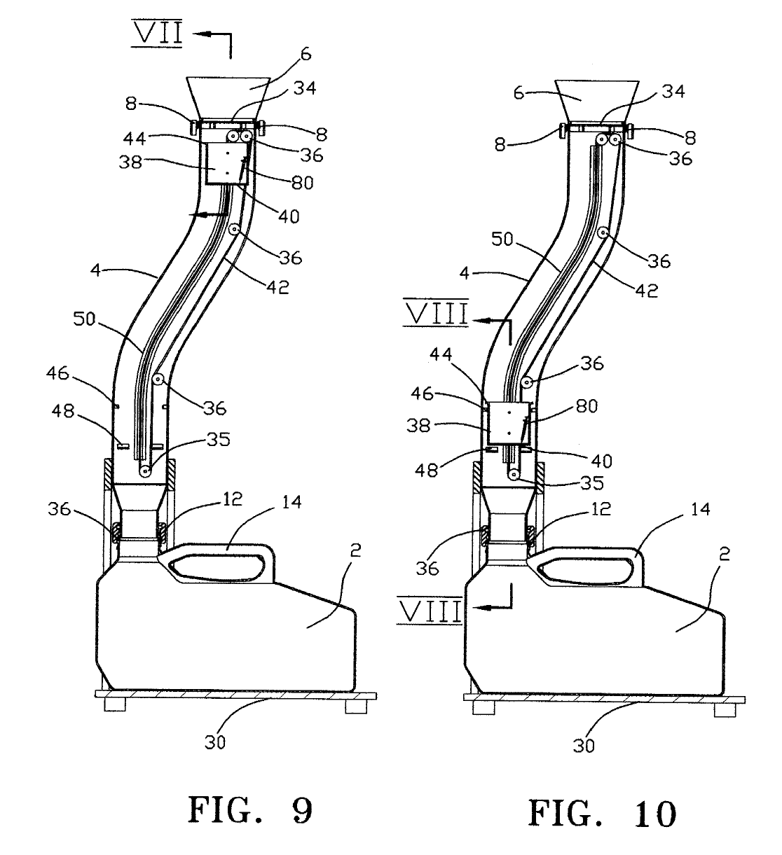

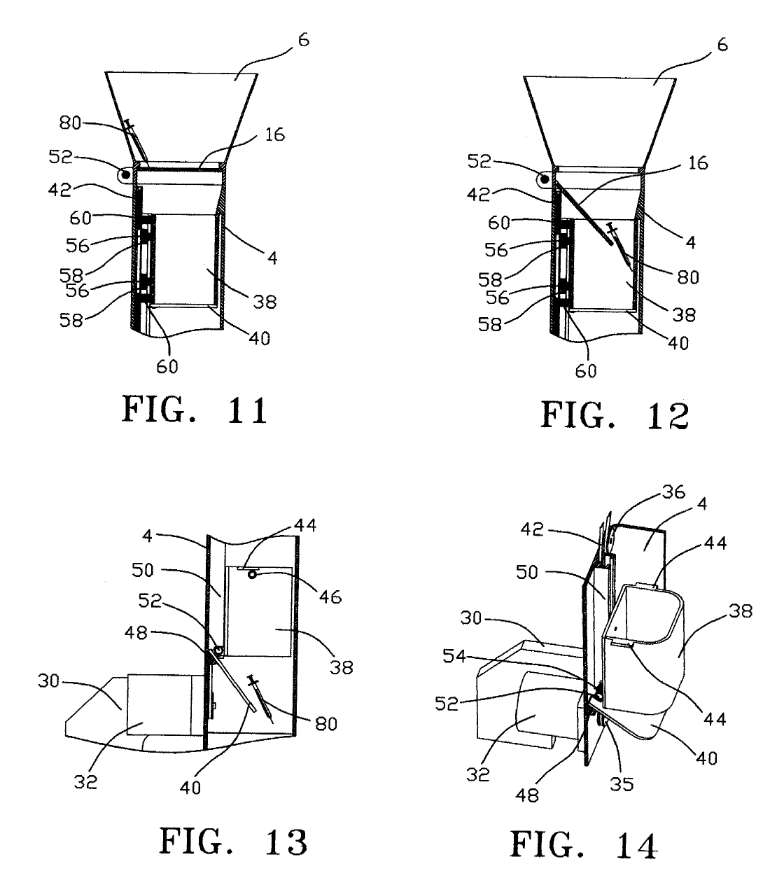

An alternative embodiment, described in regard to FIGS. 8-14, provides a mechanical conveyance to allow a positioning of mouth 6 to eliminate the need for gravity to carry the sharp 80 through the chute, as above.

In this embodiment, chute 4 includes a carriage guide 50 for carriage 38 that conveys the sharp 80 from mouth 6 to the container 2. In a preferred arrangement, the carriage guide 50 includes pulleys 36, a cable 42 and a motor 32 for driving a shaft 35 preferably arranged and mounted along a support frame 30.

As shown in FIGS. 11-12, cable holders 60 are mounted on carriage 38, which also preferably includes carriage rollers 58 mounted on shafts 56 inside the carriage guide 50. A carriage lid 40 is rotatably attached to the carriage 38 via carriage lid shaft 52 and a torsion spring 54 biases carriage lid 40 in a closed or upper position. This embodiment preferably includes carriage stoppers 44 and chute 4 contains limiters 46 and a cam 48 for opening the carriage lid 40.

In this embodiment, a medical practitioner places the sharp 80 into the mouth 6 and swing handle 8 to rotate mouth lid 16 down, allowing the sharp 80 to fall into the carriage 38 located at an receiving position. Releasing handle 8 returns mouth lid 16 to a closed position and activates motor 32, which turns shaft 35 and drives cable 42 about pulleys 36 to move the carriage 38 along the carriage guide 50 to a discharge position.

Movement of the carriage 38 to the discharge position will cause carriage lid 40 to reach cam 48, resulting in rotation about carriage lid shaft 52 to open the lid 40 to deposit the sharp 80 in collection container 2. Movement toward discharge ceases when carriage stopper 44 reaches limiter 46 and direction reverses to return carriage 38 to the receiving position wherein the carriage lid 40 gradually disengages from cam 48 to return to the closed position. When the collection container 2 fills, it can be replaced by loosening joint 12.

The invented apparatus for collecting sharp medical disposables can be placed, temporarily or permanently, close to the area where the medical procedure is performed, such as adjacent to a patients' hospital bed or under a doctor's examining table. The medical worker can readily adjust location of the mouth of the apparatus, permanently or temporarily, placing the disposal within an area of the direct reach during the medical procedure. Upon completion of the procedure, the syringe or any other sharp medical waste is placed into the large mouth opening of the apparatus and then transferred into the container.

The apparatus of the present invention can be positioned on the floor and in positions where a conventional apparatus, which typically require counter space, will not operate. The apparatus of the present invention allows for full adjustment of height and position and provides for effective quarantine of the entire sharp, not just the needle portion.

Although shown and disclosed is what is believed to be the most practical and preferred embodiments, it is apparent that departures from the disclosed configurations and methods will suggest themselves to those skilled in the art and may be used without departing from the spirit and scope of the invention. Accordingly, the present invention is not restricted to the particular constructions described and illustrated, but should be construed to cohere with all modifications that may fall within the scope of the appended claims.

What is claimed is:

1. An apparatus for quarantine of medical waste, the apparatus comprising:
a mouth located at a distal end of the apparatus;
a flexibly configured chute for receiving the waste via the mouth; and
a collection container for receiving the waste via the chute, wherein the container provides a reservoir to collect the waste.

2. The apparatus of claim 1, wherein an opening of the mouth is relatively larger than a corresponding opening of the chute.

3. The apparatus of claim 1, wherein the collection container is removable.

4. The apparatus of claim 1, further comprising a spring for moving a lid of the mouth from an open to a closed position.

5. The apparatus of claim 4, further comprising a handle for opening the mouth lid.

6. The apparatus of claim 1, wherein the flexible configuration of the chute allows for vertical and horizontal adjustment.

7. An apparatus for disposal of sharps, the apparatus comprising:
a mouth for receiving the sharps, wherein the mouth opens into a chute;
a carriage for transport of the sharps along the chute; and
a carriage guide coupled to the chute to guide the carriage from the mouth to a removable container, which collects a plurality of the sharps.

8. The apparatus of claim 7, further comprising:
a pulley;
a cable; and
a motor, wherein the pulley, cable and motor interact to move the carriage along the carriage guide between receiving and discharge positions.

9. The apparatus of claim 8, further comprising:
cable holders mounted on the carriage;
carriage rollers mounted on shafts within the carriage guide; and
a carriage lid rotatably attached to the carriage via a carriage lid shaft.

10. The apparatus of claim 9, further comprising:
a torsion spring biasing the carriage lid in a closed position.

11. The apparatus of claim 10, further comprising:
carriage stoppers, limiters and a cam that interact to overcome the bias to open the carriage lid.

12. The apparatus of claim 11, wherein, upon reaching the cam, the lid opens by rotation of the carriage lid shaft, to deposit the sharp in the collection container.

13. The apparatus of claim 7, wherein release of the handle returns the mouth lid to a closed position and activates the motor to drive the cable to move the carriage to a discharge position.

14. The apparatus of claim 7, wherein the mouth can be freely positioned without gravity assist to carry the sharp through the chute.

15. A method for avoid sticking by a medical sharp, the method comprising:
prior to a medical procedure, creating an immediately available disposal point by positioning a mouth of a sharps collection apparatus adjacent to a patient; and
disposing of the medical sharp by pushing the medical sharp into the mouth of the apparatus, thereby eliminating excess motion by the caregiver during the medical procedure.

16. The method of claim 15, further comprising operating a swing handle to rotate a mouth lid to allow the sharp to fall into a chute of the apparatus.

17. The method of claim 15, wherein a carriage of the apparatus transports the sharp to a removable collection canister.
